(19) United States
(12) Patent Application Publication
(10) Pub. No.: US 2017/0354686 A1
(43) Pub. Date: Dec. 14, 2017

(54) ADIPOSE DERIVED IMMUNOMODULATORY CELLS FOR IMMUNOTHERAPY OF RECURRENT SPONTANEOUS ABORTIONS

(71) Applicant: Creative Medical Technologies, Inc., Phoenix, AZ (US)

(72) Inventors: Thomas Ichim, San Diego, CA (US); Amit Patel, Salt Lake City, UT (US)

(21) Appl. No.: 15/617,813

(22) Filed: Jun. 8, 2017

Related U.S. Application Data
(60) Provisional application No. 62/347,898, filed on Jun. 9, 2016.

(51) Int. Cl. A61K 35/28; C12N 5/0775

(52) U.S. Cl. CPC A61K 35/28; C12N 5/0667; C12N 2500/84

(57) ABSTRACT

Disclosed are means of treating spontaneous abortion utilizing cellular populations derived from autologous adipose tissue. In one embodiment the invention teaches the collection of stromal vascular fraction from women attempting to conceive, isolation of anti-abortigenic cells from said stromal vascular fraction, storing said cells, and administering said cells in early pregnancy or when conception is desired.

### CROSS-REFERENCE TO RELATED APPLICATIONS

This application claims priority to U.S. Provisional Application No. 62/347,898, filed Jun. 9, 2016, which is incorporated herein by reference in its entirety.

### FIELD OF THE INVENTION

The invention pertains to the area of immune modulation, more specifically, the invention pertains to the use of stem cells as immune modulators, more particularly, the invention pertains to the use of adipose derived stem cells to immune modulate pathological immune responses against fetal antigens in pregnancies at risk of miscarriage.

### BACKGROUND OF THE INVENTION

Abortion is classically defined as the termination of pregnancy before the 28th gestational week characterized by expulsion of the fetus or fetal death. Abortion can be classified into early abortion (before the 12th gestational week) and late abortion (after that time point). Spontaneous abortion is the termination of pregnancy due to natural causes, such as some kind of diseases, without artificial interference. Spontaneous abortion includes accidental spontaneous abortion and recurrent spontaneous abortion (RSA), which is two or more consecutive abortions characterized by the termination of fetal development in the same gestational week. It is believed that 2-3% of pregnant women suffer from RSA.

RSAs are believed to be the result fetal chromosomal abnormalities, endocrine imbalance, anatomical abnormality of reproduction organs, bacterial infection, viral infection, blood group incompatibility between mother and fetus and environmental pollution [1]. About half of RSAs still have no known cause, and are called unexplained RSAs. Along with the deep understanding of reproductive immunology and the development of immunological assays, immunological factors are thought to be the main cause of unexplained RSAs [2].

There are several representative hypotheses about the immunological mechanism of RSA, for example: production of the blocking antibodies (BA), such as anti-paternal cytotoxic antibodies (APCA), anti-idiotypic antibodies (Ab2) and mixed lymphocyte reaction blocking antibodies (MLR-Bf) which can inhibit the attack to fetus by maternal immunological system, is inhibited due to the increased sharing of human leukocyte antigens (HLA) between the couple [3, 4]. This is in fact supported by findings of smaller placental sizes in inbred animal strains compared to outbred animals [5]. Another potential cause of RSA is overactivity of helper T cell 1 (Th1)-derived cytokines and of natural killer cells (NK) [6-8].

Since the immune recognition mechanism between pregnant woman and fetus has not been fully revealed, the immunological pathogenesis of RSA has not yet been accurately understood. No method of treatment with definite curative effect is available heretofore. Currently, one widely used method for treating immunological RSA is lymphocyte immunotherapy. Immunotherapy of RSA has been applied both in China and other countries since Taylor and Faulk infused to a patient of unexplained RSA a suspension of mixed leukocytes derived from her spouse in 1981, which was subsequently confirmed in larger trials [9]. For this type of therapy, the immunogen is lymphocytes from the spouse in most cases. The immunotherapy includes isolating lymphocytes from the spouse's venous blood for intracutaneous injection. Alternatively, the condensed leucocytes or whole blood from the spouse can also be intravenously injected. Usually, the immunization is performed every 2 weeks for a total of 2 to 4 times before pregnancy and boosted 1 to 3 times after pregnancy. Twenty years after the application of lymphocyte immunotherapy for treating RSA, a great deal of studies from China and other countries have indicated that the therapeutic effect of this therapy is not definite and the therapy has some serious adverse side effects. Most literatures on immunotherapy of RSA from 1981 to 1994.9 had been reviewed. It was found that only one of the six studies that were worthy of analysis demonstrated the effectiveness of the immunotherapy. There was no statistically significant difference between the therapy group and the control group in the other studies. In addition, the lymphocyte immunotherapy has some serious adverse side effects such as erythrocyte sensitization, thrombocytopenia and intrauterine growth retardation of fetus etc. Some diseases transmitted by blood such as AIDS may be transferred from one individual to another due to the living cells with intact nuclear materials are used in lymphocyte therapy. Accordingly, there is a need in the art for novel methods of treating immunologically associated reproductive abnormalities.

### SUMMARY OF THE INVENTION

Embodiments herein are directed to methods of reducing risk of pregnancy complications comprising administering a sufficient amount of adipose stromal vascular fraction cells capable of inhibiting anti-fetal associated maternal immune responses.

### BRIEF DESCRIPTION OF THE DRAWINGS

FIG. 1 shows a bar graph showing the effects of SVF on spontaneous resorption in mice.

### DESCRIPTION OF THE INVENTION

The current invention teaches the use of various cells and compositions derived from cells to induce immune modulation systemically, or in some cases locally in a mammal susceptible to pregnancy complications. Cells such as mesenchymal stem cells, preferably adipose derived stromal vascular fraction cells, useful for the practice of the invention cause immune modulation locally and/or systemically depending on the specific embodiment. Particularly, cells are chosen based on ability to counteract immunological abnormalities associated with RSA. Specific abnormalities include: elevated natural killer cell numbers [32], reduced number of T regulatory cells [2, 33, 34] and increased Th1/Th17 cytokines [35]. In one particular embodiment of the invention, standard SVF therapeutic protocols for autoimmunity, such as published by Ichim et al (Cell Immunol. 2010; 264(1):7-17) are applied to a patient at risk for RSA. Specifically SVF may be extracted prior to conception, and administered approximately 1-3 months after conception. Various regimens of administration may be used, however, in a preferred embodiment, 20-100 million SVF cells are administered weekly in the first month, and biweekly in the second and third month of pregnancy. In one embodiment the cells used for treatment are autologous stromal vascular fraction cells. In another embodiment SVF cells are administered while the female is trying to conceive. In another embodiment, SVF cells are administered to increase probability of successful in vitro fertilization.

In one embodiment of the invention, inflammatory and immunological abnormalities are identified in order to categorize risk of pregnancy complications, said pregnancy complications are defined as medical incidences that threaten the health of the mother or the offspring, and include RESA, preterm birth, pre-eclampsia including hemolysis elevated liver enzymes low platelets (HELP), premature rupture of the membrane, Antepartum hemorrhage including placental abruption, chorioamnionitis, Intrauterine growth restriction, placenta pravaevia, sequalae of intraamniotic infection. In one particular embodiment, levels of circulating factors are assessed in maternal plasma, based on abnormally high levels, interventions are chosen for treatment. In one particular embodiment, the methodology of Ruiz et al [36], is utilized for assessment of circulating IL-6. Specifically, plasma is analyzed in the second trimester of pregnancy and concentrations correlated with a baseline associated with non-complicated pregnancy. Within the context of the invention, other markers of inflammation may be utilized such as C reactive protein [37], In females who have higher concentration of inflammatory proteins as compared to baseline values from non-complicated pregnancies, an agent is administered to reduce inflammation. Numerous studies have demonstrated that RSA is associated with increased production of Th1 cytokines such as interferon gamma and reduced production of IL-10 [38, 39]. Furthermore, treatments that have demonstrated some signal of efficacy in RSA such as IVIG [40], G-CSF [41], and PLT [42], all have been shown to induce a Th1 to Th2 shift. Within the context of the current invention, use of stem cell mixtures, particularly adipose SVF for inducing immune modulation towards protecting the fetal allograft are envisioned. In one specific embodiment, SVF is used to extract autologous Treg, which are expanded and administered into a mammal suffering from RSA at a concentration sufficient to evoke a therapeutic response. Such concentrations may be determined by monitoring NK activity, assessing inflammatory cytokine production by peripheral blood mononuclear cells after stimulation with a mitogen or mitogenic antibody, or by assessment of T regulatory (Treg) cell numbers or activity. In one embodiment RSA patients are administered the CD4+CD25+ cells at a concentration of 50 million cells, once per month.

The invention teaches adipose tissue is an attractive alternative to bone marrow as a source of stem cells for treatment of RSA for the following reasons: a) extraction of adipose derived cells is a simpler procedure that is much less invasive than bone marrow extraction; b) Adipose tissue contains a higher content of mesenchymal stem cells (MSC) as compared to bone marrow; c) MSC from adipose tissue do not decrease in number with aging and can therefore serve as an autologous cell source for all patients; and d) adipose tissue is also a source of unique cell populations in addition to MSC that have therapeutic potential, including endothelial cells and regulatory T cells.

To date, clinical trials on adipose derived cells have all utilized ex vivo-expanded cells, which share properties with bone marrow derived MSC [1-6]. Preparations of MSC expanded from adipose tissue are equivalent or superior to bone marrow in terms of differentiation ability [7, 8], angiogenesis-stimulating potential [9], and immune modulatory effects [10]. Given the extra processing steps associated with ex vivo expansion of adipose cells, a simpler and perhaps safer procedure would be the use of primary adipose tissue-derived cells for therapy. SVF comprises the mononuclear cells derived from adipose tissue, which are acquired through a simple isolation procedure whereby fat is lipoaspirated and subjected to enzymatic digestion. Currently bench top closed systems for autologous adipose cell therapy, such as Cytori's Celution™ system [11] and Tissue Genesis' TGI 1000™ platform [12], are entering clinical trials. Although the majority of studies have focused on in vitro expanded adipose derived cells, SVF derived from whole lipoaspirate alleviates the need for extensive processing of the cells, thereby also minimizing the number of steps where contamination could be introduced. An important consideration in clinical scenerios where bulk SVF is utilized is the potential regenerative, angiogenic and immune regulatory contributions of the numerous cellular populations that are present.

The mononuclear fraction of adipose tissue, referred to as the stromal vascular fraction (SVF), was originally described as the proliferative component of adipose tissue by Hollenberg et al. in 1968 [13]. The cells comprising SVF morphologically resemble fibroblasts and were demonstrated to differentiate into pre-adipocytes and functional adipose tissue in vitro [14]. Although it was suggested that non-adipose differentiation of SVF may occur under specific conditions [15], the notion of “adipose-derived stem cells” was not widely recognized until a seminal paper in 2001, where Zuk et al demonstrated the SVF contains large numbers of mesenchymal-like stem cells (MSC-like) cells that could be induced to differentiate into adipogenic, chondrogenic, myogenic, and osteogenic lineages [16]. Subsequent to the initial description, the same group reported that in vitro expanded SVF derived cells had surface marker expression similar to bone marrow derived MSC, displaying expression of CD29, CD44, CD71, CD90, CD105/SH2, and SH3 and lacking CD31, CD34, and CD45 expression [17]. MSC are defined as adherent, non-hematopoietic cells expressing the surface markers CD90, CD105, and CD73, while lacking expression of CD14, CD34, and CD45, and having the ability to differentiate into adipocytes, chondrocytes, and osteocytes in vitro after treatment with the appropriate growth factors [18].

Adipose tissue has also been used clinically as a source of regenerative and immune modulatory MSC. Cytori is currently conducting two European clinical trials using autologous, adipose-derived mononuclear cells, of which MSC are believed to be the therapeutic population [19]. The PRECISE trial is a 36-patient safety and feasibility study in Europe evaluating adipose-derived stem and regenerative cells as a treatment for chronic cardiac ischemia. The APOLLO trial is a 48-patient safety and feasibility study in Europe to evaluate adipose-derived regenerative cells as a treatment for heart attacks [20]. Allogeneic uses of adipose derived MSC included treatment of GVHD associated liver failure [5] and steroid refractory GVHD [6, 21]. Allogeneic placenta and cord blood-derived MSC have also been used for treatment of heart failure [22] and Buerger's Disease [23], respectively. From the above-mentioned clinical trials of allogeneic MSC, graft versus host or pathological immunological reactions have not been reported. Additionally, administration of MSC intravenously, intrathecally, and intramuscularly have not been associated with ectopic tissue formation or teratoma. In addition to its stem/progenitor cell content, the SVF is known to contain monocytes/macrophages. Although pluripotency of monocytic populations have previously been described [59, 60], we will focus our discussion to immunological properties, specifically, the apparent anti-inflammatory/angiogenic activities of these cells. Initial experiments suggested that macrophage content of adipose tissue was associated with the chronic low-grade inflammation found in obese patients. This was suggested by co-culture experiments in which adipocytes were capable of inducing TNF-alpha secretion from macrophage cell lines in vitro [61]. Clinical studies demonstrated that adipocytes also directly release a constitutive amount of TNF-alpha and leptin, which are capable of inducing macrophage secretion of inflammatory mediators [62]. Interestingly, it appears from several studies in mice and humans that when monocytes/macrophages are isolated from adipose tissue, they exhibited some phenotype markers of M2 macrophages however the cells also had higher basal and induced levels of the pro-inflammatory mediators, TNF-alpha, IL-6, IL-1, MCP-1, and MIP-1 alpha, compared to levels induced by the pro-inflammatory M1 macrophages [63-65]. If indeed these adipose derived macrophages have an “M2” phenotype, they may be similar to M2 cells observed in conditions of immune suppression such as in tumors [66], post-sepsis compensatory anti-inflammatory syndrome [67, 68], or pregnancy associated decidual macrophages [69]. A recent paper suggested that it is the M2 component of SVF that is associated with enhanced survival of fat grafts that are supplemented with SVF [70]. It is estimated that the monocytic/macrophage compartment of the SVF is approximately 10% based on CD14 expression [71]. Interestingly, administrations of ex vivo generated M2 macrophages have been demonstrated to inhibit kidney injury in an adriamycin-induced model [72]. In the context of multiple sclerosis, alternatively activated, M2-like microglial cells are believed to inhibit progression in the EAE model [73]. Thus the potential M2 phenotype of adipose derived macrophages may be a mechanism of therapeutic effect of SVF cells when isolated from primary sources and not expanded.

It has been reported by us and others, that activation of T cells in the absence of costimulatory signals leads to generation of immune suppressive CD4+CD25+T regulatory (Treg) cells [74, 75]. Thus local activation of immunity in adipose tissue would theoretically be associated with reduced costimulatory molecule expression by the M2 macrophages, which may predispose to Treg generation. Conversely, it is known that Tregs are involved in maintaining macrophages in the M2 phenotype [76]. Supporting the possibility of Treg in adipose tissue also comes from the high concentration of local MSC which are known to secrete TGF-beta [77] and IL-10 [78], both involved in Treg generation [79]. Indeed numerous studies have demonstrated the ability of MSC to induce Treg cells [18, 78, 80, 81]

### Example: Syngeneic SVF Inhibits Spontaneous Abortion in the CBA×DBA Model

The established CBA×DBA mouse model of immunologically mediated spontaneous abortion [24], was utilized to assess effects of probiotic administration on resorption at day 15.

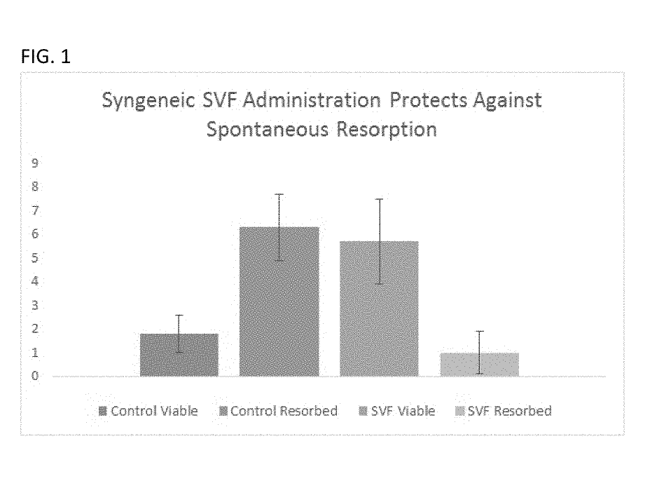

Wild type 8-10 week old virgin CBA/J female mice and 8-14 week old DBA/2J male mice were paired and vaginal plug was assessed two times a day. Day of formation of the vaginal plug was designated as day zero of pregnancy. Ten pregnant female mice were intravenously administered 500,000 syngeneic SVF cells. Another 10 mice were used as controls and treated with saline. Administration of cells was performed on day 3 of pregnancy, when animals were sacrificed and uterine horns were examined for presence of resorbed offspring. Resorption was expressed as number of resorptions/total number of formed fetuses and resorptions. See FIG. 1.

### REFERENCES

1. Garcia-Olmo, D., et al., A phase I clinical trial of the treatment of Crohn's fistula by adipose mesenchymal stem cell transplantation. Dis Colon Rectum, 2005. 48(7): p. 1416-23.
2. Stillaert, F. B., et al., Human clinical experience with adipose precursor cells seeded on hyaluronic acid-based spongy scaffolds. Biomaterials, 2008. 29(29): p. 3953-9.
3. Garcia-Olmo, D., M. Garcia-Arranz, and D. Herreros, Expanded adipose-derived stem cells for the treatment of complex perianal fistula including Crohn's disease. Expert Opin Biol Ther, 2008. 8(9): p. 1417-23.
4. Fang, B., et al., Treatment of severe therapy-resistant acute graft-versus-host disease with human adipose tissue-derived mesenchymal stem cells. Bone Marrow Transplant, 2006. 38(5): p. 389-90.
5. Fang, B., et al., Using human adipose tissue-derived mesenchymal stem cells as salvage therapy for hepatic graft-versus-host disease resembling acute hepatitis. Transplant Proc, 2007. 39(5): p. 1710-3.
6. Fang, B., et al., Favorable response to human adipose tissue-derived mesenchymal stem cells in steroid-refractory acute graft-versus-host disease. Transplant Proc, 2007. 39(10): p. 3358-62.
7. Hayashi, O., et al., Comparison of osteogenic ability of rat mesenchymal stem cells from bone marrow, periosteum, and adipose tissue. Calcif Tissue Int, 2008. 82(3): p. 238-47.
8. Noel, D., et al., Cell specific differences between human adipose-derived and mesenchymal-stromal cells despite similar differentiation potentials. Exp Cell Res, 2008. 314(7): p. 1575-84.
9. Kim, Y., et al., Direct comparison of human mesenchymal stem cells derived from adipose tissues and bone marrow in mediating neovascularization in response to vascular ischemia. Cell Physiol Biochem, 2007. 20(6): p. 867-76.
10. Keyser, K. A., K. E. Beagles, and H. P. Kiem, Comparison of mesenchymal stem cells from different tissues to suppress T-cell activation. Cell Transplant, 2007. 16(5): p. 555-62.
11. Lin, K., et al., Characterization of adipose tissue-derived cells isolated with the Celution system. Cytotherapy, 2008. 10(4): p. 417-26.
12. http://www.tissuegenesis.com/TGI%201000%20Product%20Brochure.pdf.
13. Hollenberg, C. H. and A. Vost, Regulation of DNA synthesis in fat cells and stromal elements from rat adipose tissue. J Clin Invest, 1969. 47(11): p. 2485-98.
14. Gaben-Cogneville, A. M., et al., Differentiation under the control of insulin of rat preadipocytes in primary culture. Isolation of homogeneous cellular fractions by gradient centrifugation. Biochim Biophys Acta, 1983. 762(3): p. 437-44.
15. Glick, J. M. and S. J. Adelman, Established cell lines from rat adipose tissue that secrete lipoprotein lipase. In Vitro, 1983. 19(5): p. 421-8.
16. Zuk, P. A., et al., Multilineage cells from human adipose tissue: implications for cell-based therapies. Tissue Eng, 2001. 7(2): p. 211-28.
17. Zuk, P. A., et al., Human adipose tissue is a source of multipotent stem cells. Mol Biol Cell, 2002. 13(12): p. 4279-95.
18. Prockop, D. J., Marrow stromal cells as stem cells for nonhematopoietic tissues. Science, 1997. 276(5309): p. 71-4.
19. Meliga, E., et al., Adipose-derived cells. Cell Transplant, 2007. 16(9): p. 963-70.
20. http://www.cytoritx.com/intl/products/cv_clinical_trials.html.
21. Fang, B., et al., Human adipose tissue-derived mesenchymal stromal cells as salvage therapy for treatment of severe refractory acute graft-vs.-host disease in two children. Pediatr Transplant, 2007. 11(7): p. 814-7.
22. Ichim, T. E., et al., Placental mesenchymal and cord blood stem cell therapy for dilated cardiomyopathy. Reprod Biomed Online, 2008. 16(6): p. 898-905.
23. Kim, S. W., et al., Successful stem cell therapy using umbilical cord blood-derived multipotent stem cells for Buerger's disease and ischemic limb disease animal model. Stem Cells, 2006. 24(6): p. 1620-6.
24. Bonney, E. A. and S. A. Brown, To drive or be driven: the path of a mouse model of recurrent pregnancy loss. Reproduction, 2014. 147(5): p. R153-67.

1. A method of reducing risk of pregnancy complications comprising of administering a sufficient amount of adipose stromal vascular fraction cells capable of inhibiting anti-fetal associated maternal immune responses.

2. The method of claim 1, wherein said pregnancy complications are selected from a group comprising of: women at risk for recurrent spontaneous abortions (RSA), preterm birth, low birth weight, pre-eclampsia including hemolysis elevated liver enzymes low platelets (HELP), premature rupture of the membrane, Antepartum hemorrhage including placental abruption, chorioamnionitis, Intrauterine growth restriction, placenta pravaevia, sequalae of intraamniotic infection, and cerebral palsy.

3. The method of claim 2, wherein said risk of recurrent spontaneous abortion is defined as having a higher natural killer cell activity compared to an age-matched group of women with one or more successful pregnancies.

4. The method of claim 2, wherein said risk of recurrent spontaneous abortion is defined as having a deficient T regulatory cell activity compared to an age-matched group of women with one or more successful pregnancies.

5. The method of claim 2, wherein said risk of recurrent spontaneous abortion is defined as having a higher number of circulating natural killer cells as compared to a group of age-matched women with one or more successful pregnancies.

6. The method of claim 2, wherein said risk of recurrent spontaneous abortion is defined as having a lower number of circulating T regulatory cells as compared to a group of age-matched women with one or more successful pregnancies.

7. The method of claim 2 wherein said preterm birth is defined as birth before 37 weeks of gestation.

8. The method of claim 2, said risk of preterm birth is defined as possessing an increased vaginal or systemic concentrations of: a) sialidase; b) prolidase; c) glycosyltransferase types I, II and IV; d) monocyte chemotactic protein-1; e) matrix metalloproteases I, VIII and IX; f) IP-10; g) IL-6; h) IL-1 beta; i) TNF-alpha; j) fetal fibronectin and k) thrombin-antithrombin complex; 1) Salivary estriol as compared to a group of age-matched women having one or more successful pregnancies.

9. The method of claim 2, said risk of preterm birth is defined as possessing an decreased vaginal or systemic concentrations of: a) maternal serum placental leucine amniopeptidase (P-LAP); b) IL-10; c) insulin-like growth factor-binding protein-1 (IGBP-1); d) Pregnancy associated plasma protein-A (PAPP-A); e) Corticotropin-releasing hormone (CRH) as compared to a group of age-matched women having one or more successful pregnancies.

10. The method of claim 1, wherein said stromal vascular fraction cells are obtained by the following steps; a) Using aseptic technique and with local anesthesia, the infraumbilical region is infiltrated with 0.5% Xylocaine with 1:200,000 epinephrine; b) After allowing 10 minutes for hemostasis, a 4 mm cannula attached to a 60 cc Toomey syringe is used to aspirate 500 cc of adipose tissue in a circumincisional radiating technique; c) As each of 9 syringes are filled, said syringes are removed from the cannula, capped, and exchanged for a fresh syringe in a sterile manner within the sterile field; d) Using aseptic laboratory technique, the syringe-filled lipoaspirate are placed into two sterile 500 mL centrifuge containers and washed three times with sterile Dulbecco's phosphate-buffered saline to eliminate erythrocytes; e) ClyZyme/PBS (7 mL/500 mL) is added to the washed lipoaspirate using a 1:1 volume ratio; f) The centrifuge containers are sealed and placed in a 37° C. shaking water bath for one hour then centrifuged for 5 min at 300 rcf; g) Following centrifugation, the stromal cells are resuspended within Isolyte in separate sterile 50 mL centrifuge tubes; g) The tubes are centrifuged for 5 min. at 300 rcf and the Isolyte is removed, leaving cell pellet; h) The pellets are resuspended in 40 ml of Isolyte, centrifuged again for 5 min at 300 rcf. The supernatant is again be removed; i) The cell pellets are combined and filtered through 100 μm cell strainers into a sterile 50 ml centrifuge tube and centrifuged for 5 min at 300 rcf and the supernatant removed, leaving the pelleted adipose stromal cells.

11. The method of claim 1, wherein said stromal vascular fraction cells are obtained by purifying the nucleated cell component from a lipoaspirate.

12. The method of claim 19, wherein said cells are administered at a concentration of approximately 100 million cells per dose approximately 1 month after conception.

13. The method of claim 1, wherein said adipose derived cells are cultured for expansion of mesenchymal stem cells.

14. The method of claim 13, wherein said adipose derived cells are positively selected for a marker chosen from a group comprising of: a) CD105; b) CD73; c) CD44; d) CD90; e) VEGFR2; and f) TEM-1.

15. The method of claim 13, wherein said adipose derived cells are negatively selected for markers chosen from a group comprising of: a) HLA-DR; b) CD45; and c) CD14.

16. The method of claim 13, wherein said cells are grown in DMEM media supplement with antibiotics and fetal calf serum.

17. The method of claim 16, wherein said fetal calf serum is added at a concentration of 10%.
